(19) United States
(12) Patent Application Publication
(10) Pub. No.: US 2007/0088379 A1
(43) Pub. Date: Apr. 19, 2007

(54) Minimally invasive a AAPT extirpation

(76) Inventor: Jacob Schneiderman, Kiryat-Ono (IL)

Correspondence Address: Martin MOYNIHAN;PRTSI, Inc., P.O. Box 16446, Arlington, VA, 22215, US

(21) Appl. No.: 11/290,450

(22) Filed: Dec. 1, 2005

Related U.S. Application Data
(60) Provisional application No. 60/726,618, filed on Oct. 17, 2005.

(51) Int. Cl. A61M 29/00

(52) U.S. Cl. 606/194

(57) ABSTRACT

A method for extirpating a thrombus from an aorta, comprising positioning a detachment device proximal to a thrombus inside an intact portion of an aorta moving said detachment device to contact said thrombus so as to detach said thrombus from said aortic wall and extirpating said detached thrombus from said lumen.

### RELATED APPLICATION

The present application claims the benefit of U.S. Provisional Patent Application No. 60/726,618, filed Oct. 17, 2005, the content of which is incorporated herein by reference.

### FIELD OF THE INVENTION

The present invention relates to methods for extirpating an Aortic Arch Protruding Thrombus using minimally invasive techniques.

### BACKGROUND OF THE INVENTION

Aortic Arch Protruding Thrombus (AAPT) is a unique clinical entity involving a thrombus that emerges off the aortic luminal wall along the aortic arch above the heart. AAPT is associated with life threatening occluding blood clots, herein emboli, that are shed from the AAPT into arteries of the brain, internal organs and the extremities.

Presently, AAPT is considered responsible for approximately 3% of all peripheral emboli originating from a central source. AAPT generally occurs in relatively young people that have no history of coronary or peripheral atherosclerosis, but may have high blood pressure, an undiagnosed tendency for arterial thrombosis and/or may be heavy smokers.

The pathogenesis of AAPT has been attributed to rupture of a soft shallow atherosclerotic plaque located in the aortic arch and appears to be related to the exposure of necrotic core components to the blood stream; the core components including tissue factor, PAT-1 and ox-LDL. Formation of emboli from an AAPT can be compounded by preexisting thrombophilia or a transitory pro-thrombotic state.

Accurate diagnosis of AAPT is accomplished by Transoesophageal Echocardiograph (TEE). Unfortunately, TEE is usually done after the patient has already experienced serious embolic complications.

Systemic therapy with blood thinners or anticoagulants has not prover, beneficial in preventing further emboli after the initial embolic episode. Extirpation of an AAPT is therefore a major cardiovascular surgical procedure that includes cardiopulmonary bypass, deep hypothermia and arrest of the systemic circulation, all associated with high morbidity and mortality.

In spite of the tremendous need for a simple procedure that allows rapid and relatively risk free extirpation of an AAPT, there is presently no procedure that allows AAPT extirpation without general anesthesia and procedures that have high associated morbidity and mortality.

### SUMMARY OF THE INVENTION

According to the teachings of the present invention there is provided a method for extirpating a thrombus from an aorta, comprising positioning a detachment device proximal to a thrombus inside an intact portion of an aorta, moving the detachment device to contact the thrombus so as to detach the thrombus from the aortic wall, and extirpating the detached thrombus from the lumen.

In an exemplary embodiment the method further comprises monitoring a location of the thrombus. In a further exemplary embodiment, the monitoring includes monitoring during the positioning of the detachment device.

In an exemplary embodiment, the detachment device is an expandable detachment device. According to another aspect of the present invention subsequent to the positioning, the expandable detachment device is expanded.

In an exemplary embodiment, the expanding is initiated prior to the moving. According to another aspect of the present invention the expanding is substantially a part of the moving.

In an exemplary embodiment, the expandable detachment device comprises a first inflatable balloon. According to another aspect of the present invention, inflating the inflatable balloon is subsequent to the positioning.

In an exemplary embodiment, the inflation is initiated prior to the moving. In a further exemplary embodiment the inflation is substantially a part of the moving of the detachment device to detach the thrombus.

In an exemplary embodiment, the method further includes closing a lumen of a first artery branching off the aortic lumen According to another aspect of the present invention the method includes making an incision distal to the thrombus, the incision being made into at least one of the aortic lumen and a lumen of a second artery branching off the aortic wall.

In an exemplary embodiment the method further includes proximally passing a first guide wire from the incision through the aortic lumen to a position that is proximal to the thrombus. In still another aspect of the present invention the first balloon includes an interior that communicates with a lumen of a first catheter and the method further includes passing the first catheter and the first balloon through the incision.

In an exemplary embodiment, the method additionally includes operatively associating the first guide wire with at least one of the first balloon and the first catheter. According to another aspect of the present invention, the method further includes inflating the first balloon using the first catheter.

In an exemplary embodiment the first balloon is expanded until the first balloon substantially occludes the aortic lumen. According to an additional aspect of the present invention the moving of the detachment device comprises pulling the catheter distally until the first balloon contacts the thrombus.

In an exemplary embodiment, the method includes manipulating the first balloon against the thrombus during the severing. According to an additional aspect of the present invention there is provided a method further including wherein the first catheter is employed for the manipulation.

In an exemplary embodiment the first guide wire is employed for the manipulation. According to another aspect of the present invention the method further includes deflating the first balloon. In an exemplary embodiment, the method further includes pulling the first catheter distally until a substantial portion of the first catheter is protruding from the incision.

According to an additional aspect of the present invention there is provided a method further including removing the first guide wire from the incision. In an exemplary embodiment, the method includes pulling the catheter distally and removing the first balloon from the incision.

According to another aspect of the present invention the method includes, prior to the extirpating the detached thrombus from the lumen, allowing the detached thrombus to float distally toward the incision, e.g. with the natural blood flow from the heart. In an exemplar embodiment, the method includes inserting a second guide wire through the incision and passing the second guide wire distal to the thrombus.

According to an additional aspect of the present invention the method further includes passing a second balloon through the incision and distal to the thrombus.

In an exemplary embodiment, the second balloon includes an interior that communicates with a second catheter lumen. According to an additional aspect of the present invention the method includes operatively associating the second guide wire with at least one of the second balloon and the second catheter.

In an exemplary embodiment, the method further includes inflating the second balloon in a first inflating operation. According to another aspect of the present invention the method includes pulling the second catheter distally until the second balloon is contacting the thrombus.

In an exemplary embodiment, the method further includes manipulating the second catheter while the second balloon is contacting the thrombus. According to another aspect of the present invention the method includes continuing the manipulation until the thrombus is contacting at least a portion of the incision. In an exemplary embodiment, the method includes aiding the manipulation of the second balloon with the second guide wire.

According to an additional aspect of the present invention the method includes continuing the manipulation until the thrombus is either compressed against at least a portion of the incision and/or broken into at least two portions. In an exemplary embodiment, the method further comprises removing through the incision at least one of the compressed thrombus and the at least two portions.

According to another aspect of the present invention the method further includes inspecting the aortic lumen for debris from the thrombus. In an exemplary embodiment, the method includes deflating the second balloon in a first deflating operation; and positioning the second deflated balloon proximal to the debris.

According to an additional aspect of the present invention the method further includes inflating the second balloon in a second inflating operation.

In an exemplary embodiment, the method additionally includes pulling the second catheter distally until the second balloon is contacting the debris. According to another aspect of the present invention the method further includes manipulating the second catheter while the second balloon is contacting the debris.

In an exemplary embodiment, the method includes continuing the manipulation until the debris is extirpated from the incision. According to another aspect of the present invention the second balloon is deflated in a second deflating operation. In an exemplary embodiment, the second guide wire is pulled distally out of the incision.

According to another aspect of the present invention the second catheter is pulled until a substantial portion of the second catheter is protruding from the incision and removing the second catheter from the incision.

In an exemplary embodiment, the method further includes opening the lumen of the first artery that communicates with the aortic lumen. In an exemplary embodiment, the incision is closed for example using a suture and/or a tissue clip.

According to another aspect of the present at least a portion of the first balloon comprises latex. In an exemplary embodiment the first balloon is adapted to substantially fill the lumen upon inflation.

In an exemplary embodiment, the first balloon and the first catheter together comprise at least one of an aortic balloon catheter, a Foley catheter, and a Fogerty catheter.

According to an additional aspect of the present invention the method provides that at least a portion of the second balloon comprises latex and the second balloon is adapted to substantially fill the aortic lumen upon inflation.

In an exemplary embodiment, the second balloon and the second catheter comprise at least one of an aortic balloon catheter, a Foley catheter, and a Fogerty catheter.

According to another aspect of the present invention the method provides that the closing and the opening of the lumen of the first artery branching off the aortic lumen, comprises at least one of an arterial clamp and a suture tie.

In an exemplary embodiment, the monitoring of a location of the thrombus includes using a probe placed in an esophagus, the probe adapted and positioned to monitor in a transoesophagael position, for example TEE. In an exemplary embodiment, the probe is adapted to transmit and receive ultrasound signals.

According to another aspect of the present invention the method includes transmitting and receiving ultrasound signals from the probe during the monitoring. In an exemplary embodiment, the monitoring a location of the thrombus includes using a probe placed external to the esophagus.

According to an additional aspect of the present invention the monitoring device comprises a modality selected from the group consisting of X-ray modalities (e.g., CTI) and magnetic resonance imaging (e.g., MRI) modalities.

In an exemplary embodiment, the method includes placing a radionuclide in at least one of the aortic lumen and the thrombus. In an exemplary embodiment, the monitoring is performed with a device comprising a gamma-ray detector.

According to an additional aspect of the present invention the thrombus is projecting from a portion of the arch of the aorta. In an exemplary embodiment, the thrombus is projecting proximate to, and on the same luminal portion as at least one of an innominate artery, a left carotid artery and a left subclavian artery.

According to another aspect of the present invention the thrombus is projecting proximate to, and on the luminal portion located opposite at least one of an innominate artery a left carotid artery and a left subclavian artery.

In an exemplary embodiment, the thrombus comprises a body and a stalk, the stalk connecting the body to the lumen. According to an additional aspect of the present invention the severing of the thrombus comprises severing the body from the stalk and leaving the stalk attached to the aortic wall.

According to an additional aspect of the present invention the method provides; administering medication to, without damaging the aortic wall, to cause the stalk to undergo at least one of dissolution, absorption and breakup.

In an exemplary embodiment, the thrombus comprises at least two thrombus bodies and two thrombus stalks; at least one first thrombus body and first stalk located a distance from at least one second thrombus body and second stalk.

According to still further features in the described preferred embodiments, the present invention successfully addresses the shortcomings of the presently known configurations by providing a method of safely extirpating an AAPT using minimally invasive vascular surgical technique assisted by a TEE in a surgical method that will be explained below.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The method of safely extirpation of an AAPT using minimally invasive vascular surgical technique is by way of example only, with reference to the accompanying drawings. With specific reference now to the drawings in detail, it is stressed that the particulars shown are by way of example and for purposes of illustrative discussion of the preferred method of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the methods of the invention may be embodied in practice.

FIG. 1 (prior art) is a representation of an in situ AAPT, in accordance with an embodiment of the present invention where arteries have been closed off in accordance with an embodiment of the present invention;

FIGS. 2, 3, 4 and 5 are representations of extirpation of an in situ AAPT using a minimally invasive technique, in accordance with an embodiment of the present invention.

### DESCRIPTION OF THE PREFERRED EMBODIMENTS

In broad terms, the present invention relates to a system for extirpating an AAPT using minimally invasive vascular surgical technique, in embodiments in conjunction with TEE (Transoesophageal Echocardiograph). The principles of the present invention will be better understood with reference to the drawings and accompanying descriptions.

This invention has multiple applications, only a sampling of which will be presented, the many additional applications and/or modifications to the invention for each application being known to those familiar with the art.

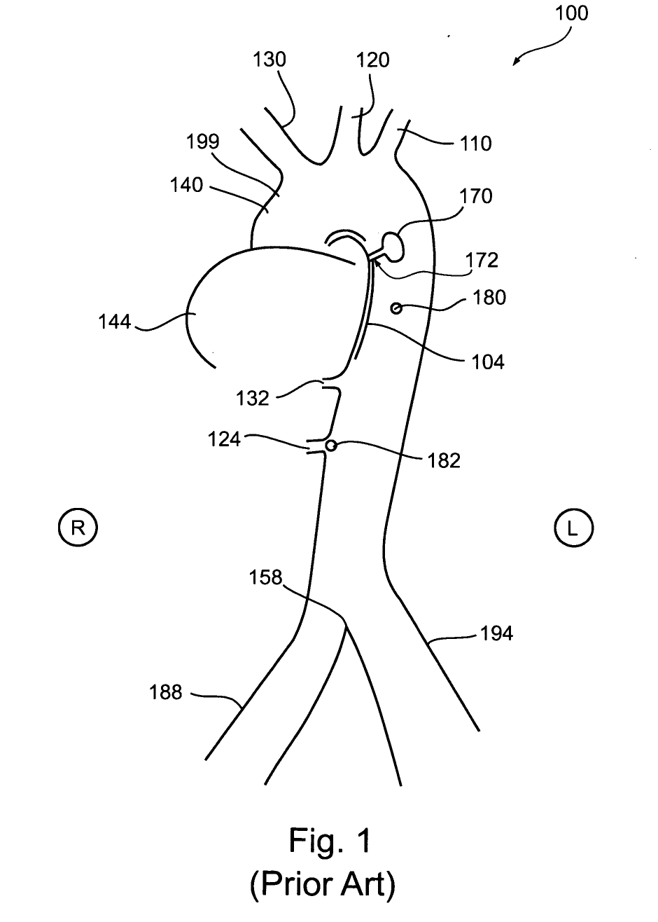

FIG. 1 is a representation of a section of an aortic 100 having an AAPT 170 projecting therefrom. Typically AAPT 170 presents in an aortic arch 140 that connects to the upper portion of a heart 144, with AAPT 170 being attached to arch 140 by a thin stalk 172 of soft organizing blood clot.

In a study of 22 cases, most AAPT 170 were located in a distal arch 199. Five were located adjacent to an innominate 130 artery, a left carotid 120 artery or a left subclavian 110 artery. (“Mobile Thromboses of the Aortic Arch Without Aortic Debris”, Theirry Laperche et al, “Circulation” 1997; 96: 288-294)

AAPT 170 typically comprises a typical thrombus composition, including fibrin, platelets, and blood cells. Due to the blood motion and beating of heart 144, AAPT 170 partially disintegrates, shedding one or more fragments as emboli 180. Emboli 180 may lodge, for example, in a celiac 132, a superior mesenteric 124 artery, or other organ-related vessels, causing tissue necrosis in associated organs, for example the spleen or intestine.

An embolus 182 is shown entering a superior mesenteric 124 artery, thereby blocking circulation to a portion of the upper intestines (not shown), likely causing ischemia and necrosis of a portion of the intestines. Necrosis of a portion of any internal organ is a medical emergency that typically requires open surgery and resection of the necrotic tissue.

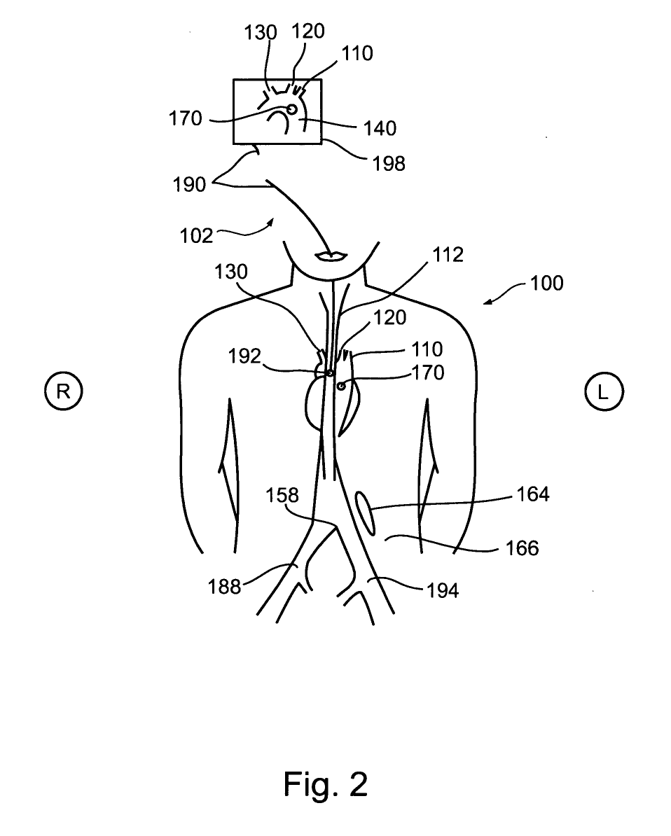

During laparotomy and following treatment of ischemnic complications, the surgeon orders a TEE 102, seen in FIG. 2. TEE 102 includes an ultrasound echo probe 192 having an ultrasound cable 190 that is passed through an esophagus 142 in a human 114. In the position shown, probe 192 demonstrates the position of AAPT 170 on a monitor 198.

The use of intra-operative, online TEE 102, connected to monitor 198 allows visualization of a luminal wall 104 of aorta 100 and AAPT 170. While TEE 102 is shown, in exemplary embodiments, other methods and/or monitoring systems and/or imaging modalities may be utilized, inter alia, intraoperative CT, MRI and nuclear imaging.

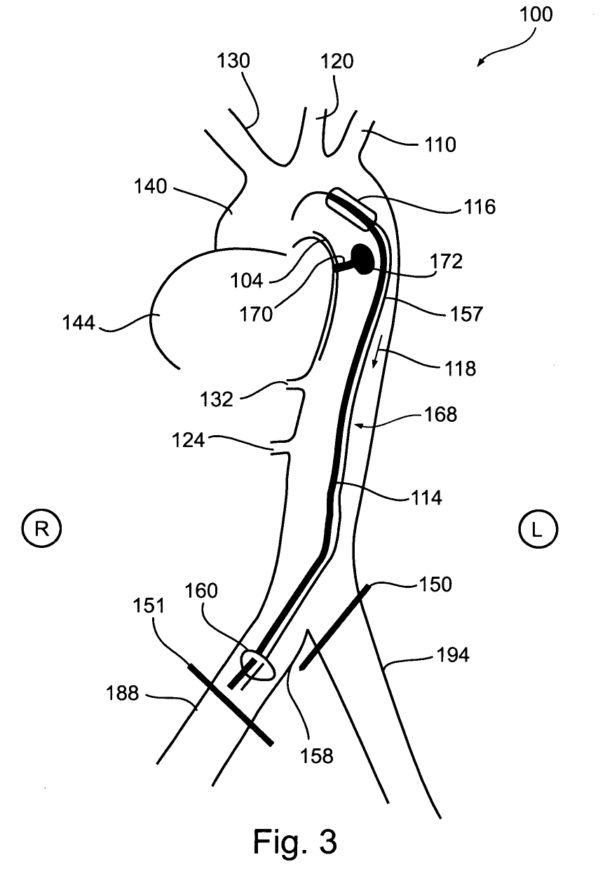

In FIG. 3, the surgeon typically places a clamp 150 on a left common iliac artery 194 and a clamp 151 on a right common iliac artery 188, thereby preventing distal embolization beyond a bifurcation 158. An incision 160 is made proximal to clamp 151.

Surgical incision 164 fosters easy access to left 194 and right 188 iliac arteries that, in turn, allow retrograde maneuverability of guide wire 1.57 and aortic balloon catheter 116. In an alternate exemplary embodiment, for example if an embolus 180 from AAPT 170 has lodged in a peripheral arty, sparing internal organs, an incision is made in the femoral artery for the guide wire 157 and balloon catheter 116 introduction.

In the iliac artery approach, a guide wire 157 is introduced and a catheter 114 having an inflatable balloon 116 is passed through an incision 160 retrograde to a direction of blood flow 118 until balloon 116 is proximal to AAPT 170.

As used herein, the terms proximal and proximally refer to positions and movement respectively toward heart 144. As used herein, the terms distal and distally refer to positions and movement respectively away from heart 144.

In an exemplary embodiment, balloon 116 and catheter 114 comprise an aortic balloon catheter 168, used for example, in stopping blood flow in an artery. In an exemplary embodiment, an aortic balloon catheter 168 is chosen in which balloon 116 has a large diameter to expand sufficiently to fill the large diameter of the lumen of aortic arch 140. Balloon 116 typically expands to at least about 3.0 centimeters in diameter.

Preferably a balloon used in implementing the teachings of the present invention is soft and has flexible walls, for example comprising latex or the like, so as to gently conform to the aortic walls to preclude damage thereto.

While an aortic balloon catheter 168 is used in prior AAPT procedures, there are additional extirpation instruments, including a Foley catheter, a Fogerty catheter, and clamps and/or bars; the type of instrument and method for use being evident to those familiar with the art.

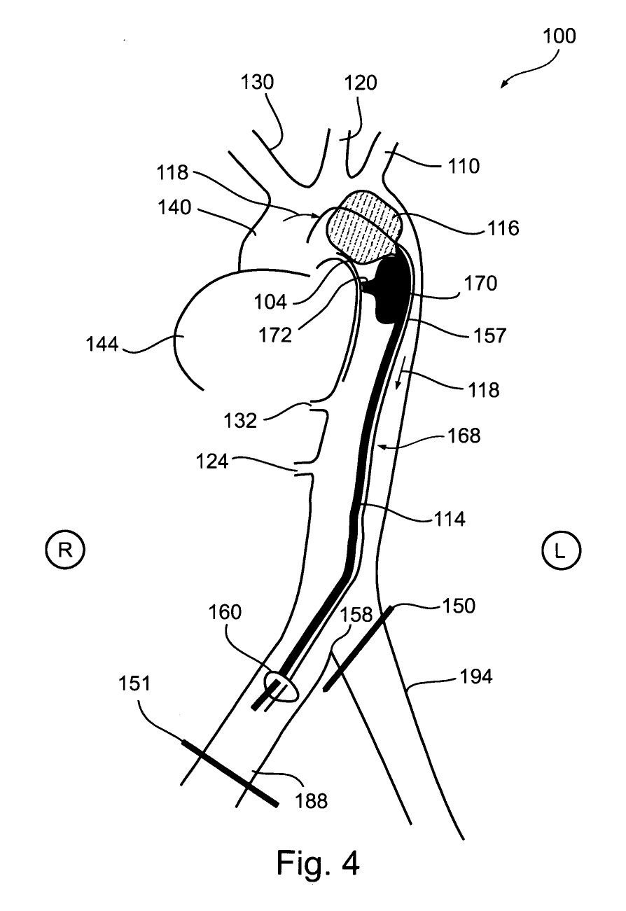

As seen in FIG. 4, balloon 116 has been introduced over guide wire 157, just proximal to AAPT 170 and inflated, for example with pressurized sterile saline in the usual way. Subsequent to inflation or during inflation, balloon 116 is gently pulled distally (direction 118) along guide wire 157 to contact AAPT 170. As a result of the contact of balloon 116 with AAPT 170 resulting from the pulling, AAPT 170 is disconnected from stalk 172. In not depicted embodiments of the present invention, the actual inflation of balloon 166 results in contact of balloon 166 with AAPT 170, leading to detachment of AAPT 170 from stalk 172.

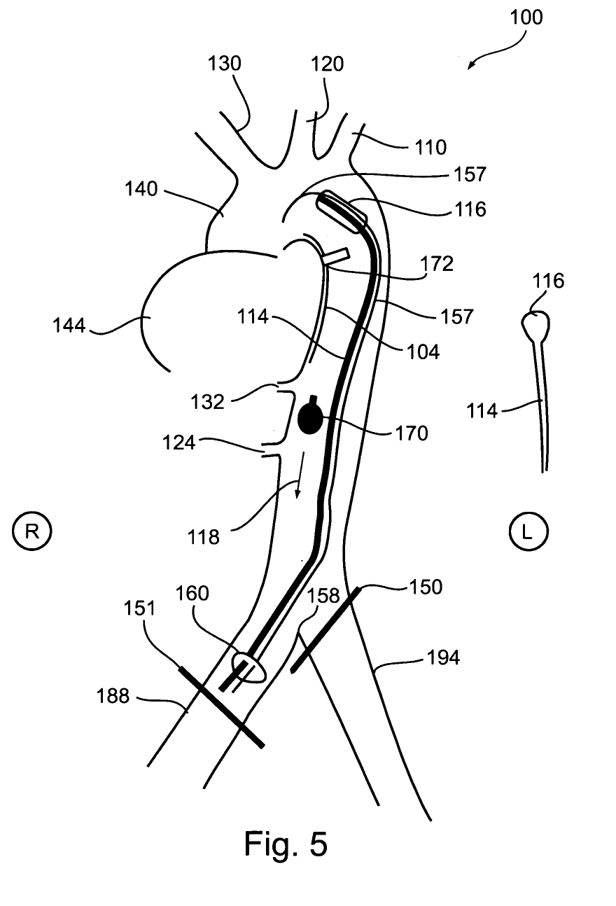

Disconnection from stalk 172 releases AAPT 170 into the lumen of aorta 100, so that AAPT 170 is free to move with blood flowing from heart 144 distally toward incision 160 (FIG. 5).

In embodiments of the invention, once released, AAPT 170 floats as one intact mass and, due to the large size of the intact mass, AAPT 170 is unlikely to block smaller arteries, for example to organs connected to celiac 132 and/or superior mesenteric 124 arteries.

In embodiments, balloon 116 is typically in an inflated state or a partially inflated state for no more than 20 seconds, no more than 15 seconds and even no more than about 10 seconds. Such a short time span lowers the chance of hemodynamic instability caused by a significant period of blood flow stoppage.

In embodiments, AAPT 170 floats to or is carried by blood flow in the aorta to incision 160 where through AAPT 170 is removed, for example with help of a Fogarty balloon catheter 152. In such cases, a Fogarty balloon is inflated proximal to AAPT 170 and removed through incision 160.

In embodiments, subsequent to removal of AAPT 170, balloon 115 and catheter 114 are removed through incision 160 in the usual way.

In embodiments, prior to removal of AAPT 170, balloon 115 and catheter 114 are removed through incision 160 in the usual way.

Following extirpation of AAPT 170, balloon 115, catheter 114 and guide wire 157, incision 160 is closed, for example with a suture or surgical clips in the usual way.

In embodiments of the invention, drugs are administered post-operatively to prevent recurrence of an AAPT 170.

Typically, assuming the patient has prothrombotic tendencies, anticoagulant therapy will be administered for life.

Additional objects, advantages, and novel features of the present invention will become apparent to one ordinarily skilled in the art upon examination of the following experimental results, which are not intended to be limiting. Additionally, each of the various embodiments and aspects of the present invention as delineated hereinabove and as claimed in the claims section below finds experimental support in the following examples.

Experimental Results

As seen in the chart, below, six cases of TEE-guided Aortic Arch Protruding Thrombus (AAPT) were successfully treated with Balloon extirpation substantially as described above. Of the six cases, five had AAPT in the distal aortic arch and one in the distal descending aorta.

Prior to AAPT, four cases were treated for abdominal ischemic complications that were related to visceral artery embolization; including two mesenteric embolectomies, one combination of mesenteric embolectomy and splenectomy; and one segmental small bowel resection. In these four cases, a right common iliac approach was utilized, while in two other cases, bilateral common femoral artery approach was used.

Thrombus material from the AAPT was retrieved in five of six cases. It is presumed that the thrombus material in the sixth case embolized into an internal iliac artery.

All patients received fill-dose anticoagulants post operatively During follow-up of up to 6 years there was no evidence of protruding thrombus recurrence, as visualized on the TEE, nor did further visceral embolizations occur.

| | | Symptomatic cardiovascular | | | | | | | | |
|---|---|---|---|---|---|---|---|---|---|---|
| | | disease & risk factors | | | | | | | | |
| | | | | | | | Thrombophilia/ | Protruding | | |
| | Sex/ | | | | | | Prothromb | thrombus | Clinical | |
| # | Age | IHD/Valv | HTN | DM | Dyslipid | Smoking | state | location | Presentation | Treatment |
| 1 | F/ | − | + | − | − | + | APCR | A: Distal | Ac. Mesenteric | Splenectomy, |
| | 50 | | | | | | Heterozygous | Desc | ischemia; | Mesentric & bilateral |
| | | | | | | | | B: Distal | Splenic&renal | fem embolectomy. |
| | | | | | | | | Arch | infarcts. | A: TEE-Guided |
| | | | | | | | | | Bilat femoral | aortic balloon |
| | | | | | | | | | artery occ. | thrombectomy |
| | | | | | | | | | | (desc) |
| | | | | | | | | | | B: TEE-Guided |
| | | | | | | | | | | Aortic Balloon |
| | | | | | | | | | | thrombectomy |
| | | | | | | | | | | (distal arch) |
| 2 | M/ | − | − | − | − | + | Factor II | Distal | Old isch. stroke. | Mesentric |
| | 62 | | | | | | Mutation | Arch | Old Bilat. | embolectomy, SB |
| | | | | | | | Heterozygous | | Femoral occ. | seg. resectior TEE- |
| | | | | | | | | | Ac on Ch. | Guided aortic |
| | | | | | | | | | Mesenteric | balloon |
| | | | | | | | | | ischemia | thrombectomy |
| 3 | F/ | − | + | − | − | + | Hyperhomo- | Distal | Iliac & Bilat. | Iliac & femoral |
| | 52 | | | | | | cysteinemia | Arch | Femoral emboli | emboloectomy |
| | | | | | | | MTHFR: | | Ac. Mesenteric | mesenteric |
| | | | | | | | Homozygous | | ischemic | emboloectomy. |
| | | | | | | | | | | TEE-Guided aortic |
| | | | | | | | | | | balloon |
| | | | | | | | | | | thrombectomy |
| 4 | F/ | − | − | − | − | + | Breast Ca | Mid Arch | Left brachial occ. | Brachia |
| | 46 | | | | | | Chemotherapy | | Left hemisph. | embolectomy. |
| | | | | | | | Hrperhomo- | | ischemic stroke | TEE-Guided aortic |
| | | | | | | | cysteinemia | | | balloon |
| | | | | | | | MTHFR: | | | thrombectomy |
| | | | | | | | Homozygous | | | |
| 5 | F/ | RHD: | + | − | − | − | Amiodarone- | Distal | Splenic infarcts | TEE-Guided aortic |
| | 45 | MS, | | | | | induced | Arch | | balloon |
| | | MR; | | | | | thyrotoxicosis | | | thrombectomy |
| | | Rapid | | | | | | | | |
| | | AF, | | | | | | | | |
| | | Left | | | | | | | | |
| | | atrial | | | | | | | | |
| | | throm | | | | | | | | |

It is expected that during the life of this patent many relevant delivery systems will be developed and the scope of the terms AAFT method is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method for extirpating a thrombus from an aorta, comprising:
positioning a detachment device proximal to a thrombus inside an intact portion of an aorta;
moving said detachment device to contact said thrombus so as to detach said thrombus from said aortic wall; and
extirpating said detached thrombus from said lumen.

2. The method according to claim 1, further comprising:
monitoring a location of said thrombus.

3. The method according to claim 2, wherein said monitoring includes monitoring during said positioning.

4. The method according to claim 1, wherein said detachment device is an expandable detachment device.

5. The method according to claim 4, further comprising:
subsequent to said positioning, expanding said expandable detachment device.

6. The method according to claim 5, wherein said expanding is initiated prior to said moving.

7. The method according to claim 5, wherein said expanding is substantially a part of said moving.

8. The method according to claim 4, wherein said expandable detachment device comprises a first inflatable balloon.

9. The method according to claim 8, further comprising:
subsequent to said positioning, inflating said inflatable balloon.

10. The method according to claim 9, wherein said inflation is initiated prior to said moving.

11. The method according to claim 9, wherein said inflation is substantially a part of said moving.

12. The method according to claim 1, further including closing a lumen of at least one artery branching off said aortic lumen.

13. The method according to claim 12, further including:
making an incision distal to said thrombus, said incision being made into at least one of;
the aortic lumen; and
a lumen of a artery branching off said aortic lumen.

14. The method according to claim 13, further including:
proximally passing a first guide wire from said incision through said aortic lumen to a position that is proximal to said thrombus.

15. The method according to claim 14, wherein said first balloon includes an interior that
communicates with a lumen of a first catheter and including;
passing said first catheter and said first balloon through said incision.

16. The method according to claim 15, further including:
operatively associating said first guide wire with at least one of:
said first balloon; and
said first catheter.

17. The method according to claim 16, including:
inflating said first balloon using said first catheter.

18. The method according to claim 17, including:
expanding said first balloon until said first balloon substantially occludes said aortic lumen.

19. The method according to claim 18, wherein said moving of said detachment device comprises pulling said catheter distally until said first balloon contacts said thrombus.

20. The method according to claim 19, including
manipulating said first balloon against said thrombus during said severing.

21. The method according to claim 20, wherein said first catheter is employed for said manipulation.

22. The method according to claim 20, including
wherein said first guide wire is employed for said manipulation.

23. The method according to claim 22, further including:
deflating said first balloon.

24. The method according to claim 23, further including:
pulling said first catheter distally until a substantial portion of said first catheter is protruding from said incision.

25. The method according to claim 24, further including:
removing said first guide wire from said incision.

26. The method according to claim 25, further including:
pulling said catheter distally; and
removing said first balloon from said incision.

27. The method according to claim 26, further including:
prior to said extirpating said detached thrombus from said lumen, allowing said detached thrombus to float distally toward said incision.

28. The method according to claim 27, further including:
inserting a second guide wire through said incision; and
passing said second guide wire distal to said thrombus.

29. The method according to claim 28, further including:
passing a second balloon through said incision and distal to said thrombus.

30. The method according to claim 29, wherein said second balloon includes an interior that communicates with a second catheter lumen.

31. The method according to claim 30, further including:
operatively associating said second guide wire with at least one of:
said second balloon; and
said second catheter.

32. The method according to claim 31, further including:
inflating said second balloon in a first inflating operation.

33. The method according to claim 32, further including:
pulling said second catheter distally until said second balloon is contacting said thrombus.

34. The method according to claim 33, further including:
manipulating said second catheter while said second balloon is contacting said thrombus.

35. The method according to claim 34, further including:
continuing said manipulation until said thrombus is contacting at least a portion of said incision.

36. The method according to claim 8, wherein at least a portion of said first balloon comprises latex.

37. The method according to claim 8, wherein said first balloon is adapted to substantially fill said lumen upon inflation.

38. The method according to claim 15, wherein said first balloon and said first catheter together comprise at least one of:
an aortic balloon catheter
a Foley catheter; and
a Fogerty catheter.

39. The method according to claim 29, wherein at least a portion of said second balloon comprises latex and said second balloon is adapted to substantially fill said aortic lumen upon inflation.

40. The method according to claim 39, wherein said second balloon and said second catheter comprise at least one of:
an aortic balloon catheter
a Foley catheter; and
a Fogerty catheter.

41. The method according to claim 2, wherein said monitoring a location of said thrombus includes using a probe placed in an esophagus, said probe adapted and positioned to monitor in a transoesophagael position.

42. The method according to claim 41, wherein said probe is adapted to transmit and receive ultrasound signals.

43. The method according to claim 42, including:
transmitting and receiving ultrasound signals from said probe during said monitoring.

44. The method according to claim 2, wherein said monitoring a location of said thrombus includes using a probe placed external to said esophagus.

45. The method according to claim 44, wherein said monitoring device comprises a modality selected from said group consisting of X-ray modalities and magnetic resonance imaging modalities.

46. The method according to claim 2, including:
placing a radionuclide in at least one of:
the aortic lumen; and
the thrombus.

47. The method according to claim 46, wherein said monitoring is performed with a device comprising a gamma-ray detector.

48. The method according to claim 1, wherein said thrombus is projecting from a portion of said arch of said aorta.

49. The method according to claim 48, wherein said thrombus is projecting proximate to, and on said same luminal portion as at least one of:
an innominate artery;
a left carotid artery; and
a left subclavian artery.

50. The method according to claim 48, wherein said thrombus is projecting proximate to, and on said luminal portion located opposite at least one of:
an innominate artery;
a left carotid artery; and
a left subclavian artery.

51. The method according to claim 48, wherein said thrombus comprises a body and a stalk said stalk connecting said body to said lumen.

52. The method according to claim 51, wherein said severing of said thrombus comprises severing said body from said stalk and leaving said stalk attached to said aortic wall.

53. The method according to claim 52, including administering medication to-prevent recurrence of said thrombus.

54. The method according to claim 48, wherein said thrombus comprises at least two thrombus bodies and two thrombus stalks; at least one first thrombus body and first stalk located a distance from at least one second thrombus body and second stalk.
